(19) United States
(12) Patent Application Publication
(10) Pub. No.: US 2015/0113681 A1
(43) Pub. Date: Apr. 23, 2015

(54) COMPOSITION AND METHOD FOR ENHANCING PLANT TRANSFORMATION

(71) Applicant: Heng Zhong, Durham, NC (US)

(72) Inventor: Heng Zhong, Durham, NC (US)

(73) Assignee: SYNGENTA PARTICIPATIONS AG, Basel (CH)

(21) Appl. No.: 14/060,898

(22) Filed: Oct. 23, 2013

(51) Int. Cl. C12N 15/82

(52) U.S. Cl. CPC C12N 15/8274
USPC 800/278; 435/418; 435/413; 435/415; 435/411; 435/468

(57) ABSTRACT

The invention includes transformation selection medias and methods, including transformation selection media comprising a negative selection agent and containing differing amounts of carbohydrate during incubation of transformed cells during the selection process, including providing an amount of carbohydrate in a transformation selection media and culturing transformed cells therein for a period of time in an incubation step followed by transferring the transformed cells into transformation selection media comprising a negative selection agent and having an amount of carbohydrate that differs from the amount of carbohydrate used in the previous transformation selection media and incubating the cells for another period of time in a second incubation step. Additional incubation steps may be included, wherein the carbohydrate content of the transformation selection media in each step may be different from the carbohydrate content of the transformation selection media used in one or more of the previous incubation steps.

### FIELD OF THE INVENTION

The present invention relates to plant tissue culture selection media comprising a negative selection agent and methods designed to more efficiently obtain transgenic plant cells and regenerated plants therefrom.

### BACKGROUND

The invention relates to the production of transgenic plants involving plant cells or tissue being transformed with a gene of interest and then regenerated into whole plants. Representative current methods for transforming plants by introducing a gene of interest can require that the cells or tissue be maintained in plant culture media for several weeks to effect selection or to support sufficient tissue growth. Many commercially important plants, plant cells, or plant tissues are difficult to maintain in tissue culture, and this poses a limitation on the number of transgenic plants that can successfully be regenerated from tissue culture.

Thus, there is a continuing need to provide plant transformation media that enhance effective selection and growth of transformed tissue/cells to survive in the media during the transformation/regeneration process. The present invention includes a composition and method that increases the overall efficiency of the transformation process.

### SUMMARY

The invention relates to a composition and method for genetically transforming a plant cell, tissue or other suitable explant and regenerating a transformed plant therefrom. In accordance with the presently disclosed subject matter, the method provides for introducing a nucleic acid into the genome of a plant cell wherein differing amounts of a compound or compounds which provide a carbohydrate and/or osmotic source, such as sucrose, glucose, fructose, maltose, galactose, and dextrose, is included in the transformation selection media comprising a negative selection agent. The invention includes contacting plant cell, tissue or explant with a transformation selection media comprising a negative selection agent and comprising differing amounts of a carbohydrate, such as sucrose, sufficient to enhance the efficiency of selection and/or transformation, and/or the survivability of the plant cell, tissue or explant compared to the transformation efficiency when the transformation selection media does not include differing amounts of a carbohydrate. The transformation selection media discussed herein, unless otherwise stated, comprises a negative selection agent.

By way of example, the invention includes transformation selection media having differing amounts of carbohydrate during incubation of cells, including providing an amount of carbohydrate in the transformation selection media and culturing transformed cells therein for a period of time in an incubation step, followed by transferring the transformed cells into transformation selection media having an amount of carbohydrate that differs from the amount of carbohydrate used in the previous transformation selection media and incubating the cells for another period of time in a second incubation step.

By way of further example, the invention includes transformation selection medias and methods, including transformation selection media containing differing amounts of carbohydrate during incubation of transformed cells during the selection process, including providing an amount of carbohydrate in a transformation selection media and culturing transformed cells therein for a period of time in an incubation step followed by transferring the transformed cells into transformation selection media having an amount of carbohydrate that differs from the amount of carbohydrate used in the previous transformation selection media and incubating the cells for another period of time in an incubation step. Additional incubation steps may be included, wherein the carbohydrate content of the transformation selection media in each step may be different from the carbohydrate content of the transformation selection media used in one or more of the previous incubation steps.

By way of further example, one of the incubation steps of the present invention can include the use of a transformation selection media that does not contain a negative selection agent.

Using differing amounts of carbohydrate in the selection media can also include having at least three transformation selection media incubation steps. The first step in the three incubation steps comprises using a transformation selection media comprising an amount of carbohydrate and incubating transformed cells therein for a period of time. A second step includes a transformation selection media containing a carbohydrate in an amount different from the amount of carbohydrate in the first transformation selection media and transferring the transformed cells incubated in the transformation media of the first step to the transformation selection media of the second step and incubating for a time, and followed by a third step comprising a transformation selection media containing an amount of carbohydrate in an amount different from the amount the transformation selection media used in the second step, and transferring the transformed cells incubated in the transformation selection media of the second step into the transformation selection media of the third step and incubating for a time. Transformation selection media incubations are typically followed by incubating transformed cells into a regeneration media that includes about 20 to 30 mg/L carbohydrate without a selection agent.

The invention includes transformation selection media having differing amounts of carbohydrate during incubation of cells, including using an amount of carbohydrate in the transformation selection media and incubating transformed cells therein for a period of time in an incubation step followed by transferring the transformed cells into transformation selection media having an amount of carbohydrate that differs from the amount of carbohydrate used in this previous transformation selection media and incubating such cells for another period of time in an incubation step, wherein the amount of carbohydrate in the first incubation media is less than the amount of carbohydrate in the transformation selection media used in an incubation step.

A plant transformation selection media used to transform a plant cell, tissue or other suitable explant to generate a plant therefrom, comprising a carbohydrate energy source in the amount of 1 g/L to about 15 g/L and a negative selection agent in an amount effective to select for transformants, wherein said the carbohydrate energy source increases plant transformation frequency compared to the plant transformation frequency obtained when using a carbohydrate energy in the transformation selection media in an amount greater than 15 g/L

The invention includes any number and combinations of incubation steps, wherein the incubation steps use transformation selection media having differing amounts of carbohydrate.

The media described herein can be liquid, solid or semi-solid, and a carbohydrate can be included in any of the particular media used during the “transformation process”, e.g., the inoculation, co-cultivation, selection, shoot induction, elongation, regeneration or rooting media. The compounds of the invention can also be used in one or more of such particular media used during the “transformation process.” The carbohydrate and amount used in the media may vary between different species and cultivars within a species (Kumria et al. 2001; Sahoo et al. 2011; Jain et al. 1997; Ren et al. 2010; Das and Joshi 2010; Swedlund and Locy 1993; Geng et al. 2008; Xu et al. 2009; Soo 2013; Joersbo et al. 2003; Godo et al. 1996; Da Silva, 2004)

The present invention also provides a method for transforming dicotyledonous and monocotyledonous plant tissue, selecting transformed cells and regenerating fertile transgenic plants therefrom comprising differing amounts of a carbohydrate in the plant selection media during the incubation steps of the transformation selection process.

The present invention also provides using differing amounts of a carbohydrate in one or more plant selection media during incubation of transformed cells during the transformation process, sufficient to enhance the efficiency of selection and/or transformation, and/or the survivability of the plant cell, tissue or explant, compared to the transformation efficiency of tissue or explant where the carbohydrate is included in a constant amount in the plant selection media throughout the incubation of transformed cells on selection media.

In accordance with the presently disclosed subject matter, the method provides for introducing a nucleic acid into the genome of a plant cell wherein a reduced amount of a carbohydrate such as sucrose, glucose, fructose, maltose, galactose, and dextrose, is included in the transformation selection media. The invention includes placing plant cells, tissue or explant in contact with a transformation selection media comprising a reduced amount of carbohydrate and incubating such cells, tissue or explant for a period of time followed by transferring these cells into transformation selection media having an amount of carbohydrate greater than the carbohydrate used in this previous incubation step, thereby enhancing the efficiency of selection and/or transformation, and/or the survivability of the plant cell, tissue or explant compared to such transformation efficiency of tissue or explant wherein a reduced amount of carbohydrate is not included in the transformation media.

In one embodiment of the invention, the incubation step using selection media comprising a reduced amount of carbohydrate immediately precedes the last incubation step of the transformation selection process.

The present invention further provides plant transformation media comprising differing amounts of a compound or compounds which provide a carbohydrate and osmotic source, such as sucrose, glucose, fructose, maltose, galactose, and dextrose. The media can be liquid, solid or semi-solid, and a compound or compounds which provide a carbohydrate and/or osmotic source can be included in any of the particular media used during the “transformation process”, e.g., the inoculation, co-cultivation, selection, shoot induction, elongation, regeneration or rooting media. The compounds of the invention can also be used in one or more of such particular media used during the “transformation process.” Preferential carbohydrate and/or osmotic sources can vary between different species and even cultivars within a species.

The present invention also provides a method for transforming dicotyledonous and monocotyledonous plant tissue, selecting transformed cells and regenerating fertile transgenic plants therefrom comprising a reduced amount a carbohydrate in at least one of the plant selection media during the transformation process.

The invention further includes a method of identifying a transformed plant cell comprising:
a. isolating a explant suitable for transformation;
b. combining the explant with a gene to produce transformed plant cells;
c. culturing the transformed plant cells in a plant transformation selection media wherein the selection media contains a reduced level of carbohydrate energy source and a negative selection agent and incubating the cells for a period of time;
d. transferring the cells incubated in step (c) to transformation selection media containing a negative selection agent and an amount of carbohydrate energy source greater than the carbohydrate energy source contained in the plant tissue culture media of step (c) and incubating the cells for a period of time;
e. identifying the transformed cells incubated in the transformation selection media in step (d).

The invention also includes a method of producing a transformed plant comprising:
a. isolating a explant suitable for transformation;
b. combining the explant with a gene to produce transformed plant cells;
c. culturing the transformed plant cells in a plant transformation selection media wherein the selection media contains a reduced level of carbohydrate energy source and a negative selection agent and incubating the cells for a period of time;
d. transferring the cells incubated in step (c) to transformation selection media containing a negative selection agent and an amount of carbohydrate energy source greater than the carbohydrate energy source contained in the plant tissue culture media of step (c) and incubating the cells for a period of time;
e. identifying the transformed cells incubated in step (d); and
f. regenerating at least one transformed cell identified in step (e) to produce a transformed plant.

### DEFINITIONS

“Transformation media” or “plant transformation media,” as used herein, refers to the plant tissue culture media, whether liquid, solid or semi-solid, used during the process of the transformation of plant cells, tissues, parts or other plant tissue explants and subsequent regeneration of whole, transgenic plants therefrom. Depending upon the plant species being transformed and the transformation process being used, the transformation media can include, but is not limited to, the isolation media, inoculation medium, induction media, recovery media, selection media, regeneration media and/or rooting media.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (De Blaere et al., 1987) and particle bombardment technology (Klein et al., 1987; U.S. Pat. No. 4,945,050), however, many other methods of transformation of cells are known to the art. Whole plants may be regenerated from transgenic cells by methods well known to the skilled artisan (see, for example, Fromm et al., 1990).

The term “transferring” cells into media refers to moving cells from one media to the next, but can also mean changing, exchanging, or any other way of varying the media in which the cells are incubated or cultured.

Transformation frequency (TF),” as used herein, refers to the percentage of transgenic events produced per total of explants or the percentage of transgenic plants produced per total of explant. This percentage can be calculated based upon the weight of the explant material, as in the case of callus transformation, or the amount of the explant material, as in the case of immature embryo transformation.

Transformation efficiency as used herein refers to the number of transgenic events generated per timed effort, e.g., human hours invested in generating transgenic events, which can be determined by TF and “escape rate.” There may be different methods for determining transformation efficiency and transformation frequency. The manner of calculating efficiency and frequency is not critical to the invention.

“Survivability” of a plant cell, tissue, part or other explant during the transformation and regeneration process, as used herein, refers to the ability of the cell, tissue, part or other explant to flourish in the transformation media with little or no browning or other disadvantageous characteristics that limit its ability to continue to divide and grow in the media.

An “event,” as used herein, refers to a recombinant plant produced by transformation and regeneration of a plant cell or tissue with heterologous DNA, for example, an expression cassette that includes a gene of interest. The term “event” refers to the original transformant and/or progeny of the transformant that include the heterologous DNA. The term “event” also refers to progeny produced by a sexual outcross between the transformant and another corn line. Even after repeated backcrossing to a recurrent parent, the inserted DNA and the flanking DNA from the transformed parent is present in the progeny of the cross at the same chromosomal location. The term “event” also refers to DNA from the original transformant comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA. Normally, transformation of plant tissue produces multiple events, each of which represent insertion of a DNA construct into a different location in the genome of a plant cell. Based on the expression of the transgene or other desirable characteristics, a particular event is selected.

A “transgenic plant” is a plant having one or more plant cells that contain a heterologous DNA sequence.

An “escape,” as used herein, refers to a plant, a plant cell, or plant tissue that survives the selection process without having the gene encoding for resistance to the selectable marker stably transformed into the genome of said plant. An “escape frequency,” as used herein, refers to the percentage of escape events produced per total of explants or the percentage of escape plants produced per total of explant. Reduction in escape rate leads to increased transformation efficiency.

“Regeneration frequency,” as used herein, refers to the percentage of the number of callus that produced plant(s) per total number of survived callus to regeneration medium.

“Plant stress condition,” as used herein, refers to less than optimal conditions necessary for maintaining healthy growth or maintenance of plant cells or tissue in plant transformation media, such as by repeated media transfers, limiting nutrients (including water and light), or less than optimal quality of plant tissue or cells such as by wounding or excessive handling. This list is not intended to be exclusive of other stress conditions known to those of ordinary skill in the art.

“Explant,” as used herein, refers to a piece or pieces of tissue. Explant tissue can be a part of a plant, such as immature embryos, leaves meristems, or can be derived from a portion of the shoot, leaves, immature embryos or any other tissue of a plant.

The terms “heterologous” and “exogenous,” as used herein, refer to a nucleotide sequence that originates from a source foreign to the particular host cell or, if from the same source, is modified from its original form. The terms also include non-naturally occurring, multiple copies of a naturally occurring DNA sequence. Thus, the terms refer to a DNA segment that is foreign to the host cell, or naturally occurring in the host cell but in a position or form within the host cell in which the element is not ordinarily found in nature.

The term “recombinant,” as used herein, refers to any gene or DNA segment that is introduced into a recipient cell, regardless of whether a similar gene might already be present in such a cell. The type of DNA included in the recombinant DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

The term “carbohydrate” refers to any carbon based compound, or an analog thereof, within a growth media required for plant cell and tissue proliferation. These carbon based compounds include, but are not limited to: monosaccharide, disaccharide and polysaccharide, such as sucrose, glucose, galactose, fructose, maltose, sorbital, dextrose, other simple sugars, glycogen, and soluble starches, and are also individually referred to herein as a carbohydrate energy source.

The term “transformed” refers to cells that have been selected and regenerated on a selection media following transformation.

The term “analog,” as used herein, refers to a structural chemical derivative of a parent compound.

The term “positive effect,” as used herein, refers to any increase in the efficiency of the transformation used. The positive effect may be as little as a fractional increase or may be an increase of several fold.

### DETAILED DESCRIPTION

A major problem inherent in transformation systems is that the process can be inefficient and extremely labor intensive. The presently disclosed subject matter provides an improved transformation selection method broadly applicable to a wider variety of plant genotypes. The presently disclosed subject matter provides an improved transformation selection method by differing the amount of one or more carbon based compounds such as carbohydrates within the plant tissue culture selection media that can increase transformation frequencies. The present invention provides an improved transformation selection media that has application to crop-species and varieties that are recalcitrant or difficult to transform.

The transformation selection media discussed herein, unless otherwise stated, comprises a negative selection agent.

Many crops are transformed by inoculating plant tissue with Agrobacterium tumefaciens; maintaining these cultured cells on transformation selection media for several weeks to effect selection for the growth of the rare, stably transformed cells; and then regenerating transgenic plantlets from the undifferentiated selected cells. One problem inherent in Agrobacterium-based transformation systems is that Agrobacterium tumefaciens do not transform efficiently. Another problem in the Agrobacterium tumefaciens transformation systems is that a large proportion of the shoots regenerated are not transformants, but “escapes” from selection. These common problems in plant transformation—efficiency and escapes—can limit transgenic plant production using Agrobacterium-mediated transformation, with effects ranging from the moderate to the severe, depending on the crop and the cultivar in question.

The present invention includes the use of a carbohydrate, also referred to herein as “carbohydrate energy source,” such as sucrose, glucose, fructose, maltose, galactose, and dextrose to enhance transformation efficiency, frequency, and/or transformed cell survivability. This list of compounds which increase transformation efficiency and frequency is intended to be exemplary and not comprehensive. Other compounds may be apparent to those skilled in the art and may be substituted here. The effect of differing sucrose levels on the frequency of generating independent stable transgenic events in corn at 5 g/L and 10 g/L concentrations were investigated. By way of example and not limitation, 5 and 10 g/L of sucrose in the bialaphose selection media first round of selection (S1), transformation frequency increased by 3.1 and 2.2 fold to 23.5% and 16.9%, respectively in Zea mays (corn) compared to the control (20 g/L, 7.6%) (Table 1). The regeneration frequency increased from 37.4% in control (20 g/L sucrose) to more than 65% in treatment (5 and 10 g/L sucrose in S1 bialaphose selection, while the escape rate decreased from 11.8% in control to 2.4% in treatment (5 g/L sucrose). In glyphosate selection, sucrose at the 5 and 10 g/L concentrations in second round selection (S2) followed by sucrose at 20 g/L concentration at the third round selection (S3) significantly increased the frequency of independent stable transgenic events, from 4.8% (negative control with a standard sucrose concentration of 20 g/L) to 29.5% and 18.3%, respectively, and the regeneration frequency increased from 7.9% to 87.4% and 86.4%, respectively (Table 2). Sucrose at 5 g/L resulted in the highest increase of transformation efficiency, thus increasing frequency of stable transgenic events.

The effect of sucrose levels in transformation selection media was determined in sugarcane. Referring to Table 3, the use of a reduced amount of sucrose in 51 (5 g/L) followed by 20 g/L in the S2 transformation selection media increased TF events by 7.14%.

The present invention includes a plant transformation selection media, comprising a carbohydrate in the amount of 1 g/L to about 15 g/L, wherein said the carbohydrate increases plant transformation frequency compared to the plant transformation frequency obtained when using a carbohydrate in the transformation selection media in an amount greater than 15 g/L.

The present invention includes a plant transformation selection media, comprising a carbohydrate in the amount of 1 g/L to about 19 g/L, wherein said the carbohydrate increases plant transformation frequency compared to the plant transformation frequency obtained when using a carbohydrate in the transformation selection media in an amount greater than 19 g/L.

Thus, the present invention includes reducing the amount of carbohydrate in at least one plant transformation media containing a negative selection agent during the selection process compared to the amount of carbohydrate currently understood by those skilled in the art as most effective. The reduced amount of carbohydrate increases the efficiency of transformation of a plant explant and/or the efficiency of selection of a transgenic plant cell during the transformation process.

The present invention also includes a method for introducing a nucleic acid sequence into the genome of a monocotyledonous or dicotyledonous plant, plant cell, or plant tissue and regenerating a transformed plant therefrom, comprising culturing the plant cell on at least one plant transformation selection media comprising a reduced amount of carbohydrate, such as sucrose, glucose, maltose, galactose, and dextrose and incubating for a period of time prior to contacting selected transformed cells to regeneration media. According to one aspect of the invention, a reduced amount of carbohydrate in the transformation selection media is from about 1 g/L to about 15 g/L, from about 2.5 g/L to about 12.5 g/L, or from about 5 g/l to about 10 g/L.

The invention includes transformation selection media having differing amounts of carbohydrate during incubation of transformed cells, including using 5 g/L, 6 g/L, 7 g/L, 8 g/L, 9 g/L, 10 g/L, 11, g/L, 12 g/L, 13 g/L 14 g/L or 15 g/L carbohydrate in the transformation selection media and incubating transformed cells therein for a period of time in an incubation step followed by transferring the transformed cells into transformation selection media having an amount of carbohydrate that differs from the amount of carbohydrate used in this previous transformation selection media and incubating such cells for another period of time in a second incubation step, wherein the amount of carbohydrate in the first incubation media is less than the amount of carbohydrate in the transformation selection media used in the second incubation step

The invention includes transformation selection media having differing amounts of carbohydrate during incubation of transformed cells, including using 5 g/L, 6 g/L, 7 g/L, 8 g/L, 9 g/L, 10 g/L, 11, g/L, 12 g/L, 13 g/L 14 g/L or 15 g/L carbohydrate in the transformation selection media and incubating transformed cells therein for a period of time in an incubation step followed by transferring the transformed cells into transformation selection media having an amount of carbohydrate that differs from the amount of carbohydrate used in this previous transformation selection media and incubating such cells for another period of time in a second incubation step, wherein the amount of carbohydrate in the transformation selection media used in the second incubation step is at least 16 g/L, 17 g/L, 18 g/L, 19 g/L, 20 g/L, 21 g/L, 22, g/L 23 g/L, 24 g/L, 25 g/L, 26 g/L, 27 g/L, 28, g/L 29 g/L or 30 g/L.

The present invention further includes an effective amount of carbohydrate that is from about 1 g/L to about 15 g/L, 2.0 g/L to about 15 g/L, 2.5 g/L to about 15 g/L, 3 g/L to about 15 g/L, 3.5 g/L to about 15 g/L, 4.0 g/L to about 15 g/L, 4.5 g/L to about 15 g/L, 5.0 g/L to about 15 g/L, 5.5 g/L to about 15 g/L, 6.0 g/L to about 15 g/L, 6.5 g/L to about 15 g/L, 7.0 g/L to about 15 g/L, 7.5 g/L to about 15 g/L, or 8/0 g/L to about 15 g/L.

The present invention further includes an effective amount of carbohydrate that is from about 1 g/L to about 12.5 g/L, 2.0 g/L to about 12.5 g/L, 2.5 g/L to about 12.5 g/L, 3 g/L to about 12.5 g/L, 3.5 g/L to about 12.5 g/L, 4.0 g/L to about 12.5 g/L, 4.5 g/L to about 12.5 g/L, or 5.0 g/L to about 12.5 g/L.

The present invention further includes an effective amount of carbohydrate that is from about 1 g/L to about 10.0 g/L, 2.0 g/L to about 10.0 g/L, 2.5 g/L to about 10.0 g/L, 3 g/L to about 10.0 g/L, 3.5 g/L to about 10.0 g/L, 4.0 g/L to about 10.0 g/L, 4.5 g/L to about 10.0 g/L, or 5.0 g/L to about 10.0 g/L.

The present invention further includes an effective amount of carbohydrate that is from about 1 g/L to about 8.0 g/L, 2.0 g/L to about 8.0 g/L, 2.5 g/L to about 8.0 g/L, 3 g/L to about 8.0 g/L, 3.5 g/L to about 8.0 g/L, 4.0 g/L to about 8.0 g/L, 4.5 g/L to about 8.0 g/L, or 5.0 g/L to about 8.0 g/L.

In one embodiment of the invention, any effective amount of carbohydrate in the transformation selection media as disclosed herein may be used in combination with an amount of bialaphos selection agent in the transformation selection media. The amount of bialaphos selection agent that may be used in the transformation selection media is about 5 mg/L to about 7.5 mg/L.

Carbohydrate levels may be reduced in plant transformation media at various individual steps or in one or more of the steps of the transformation process in different plant species to optimize its use for the particular plant species. The reduction of sucrose in a plant transformation media is beneficial during the selection stages of transformation where the plant tissues are exposed to negative selection agents, specifically herbicides. These herbicides include but are not limited to BASTA®, bialaphos, phosphinothricin, glufosinate, LIBERTY®, TOUCHDOWN®, ROUNDUP®, butafenacial, mesotrione, norflorazon, and glyphosate. These media are standard in transformation laboratories across the industry. Recipes for these media are well known to the skilled practitioner.

As described herein, the reduction of a compound or compounds which provide a carbohydrate energy source and/or osmotic source, such sucrose, glucose, fructose, galactose, maltose, dextrose, in plant transformation selection media can advantageously be used with any plant, including dicotyledonous and monocotyledonous plants. Although various transformation systems are well known to those skilled in the art, a brief description of the process is provided below.

Typically, to initiate a transformation process in accordance with the presently disclosed subject matter, it is first desirable to select the genetic components desired to be inserted into the plant cells or tissues. Genetic components can include any nucleic acid that is introduced into a plant cell or tissue using the method according to the presently disclosed subject matter. Genetic components can include non-plant DNA, plant DNA, or synthetic DNA.

Approaches for preparing plasmids or vectors containing the desired genetic components are well known in the art. Vectors typically comprise a number of genetic components, including but not limited to regulatory elements such as promoters, leaders, introns, and terminator sequences. Regulatory elements are also referred to as cis- or trans-regulatory elements, depending on the proximity of the element to the sequences or gene(s) they control. These methods are well known to those of ordinary skill in the art and have been reported (see, for example, Sambrook et al., Molecular Cloning: A Laboratory Manual, Second Edition, Cold Spring Harbor Laboratory Press, Cold Spring Harbor, N.Y., 1989).

The present invention can be used with any suitable plant transformation plasmid or vector containing a selectable or screenable marker and associated regulatory elements, along with one or more nucleic acids (a structural gene of interest) expressed in a manner sufficient to confer a particular desirable trait. Preferably, the selectable marker is an herbicide resistance gene. Examples of suitable structural genes of interest envisioned by the presently disclosed subject matter can include, but are not limited to, genes for insect or pest tolerance, herbicide tolerance, heterologous enzyme expression, genes for quality improvements such as yield, nutritional enhancements, environmental or stress tolerances, or any desirable changes in plant physiology, growth, development, morphology, or plant product(s).

Exemplary nucleic acids that can be introduced by the methods encompassed by the presently disclosed subject matter include, for example heterologous, exogenous, and/or recombinant nucleic acid sequences, as defined herein.

In light of the present disclosure, numerous other possible selectable or screenable marker genes, regulatory elements, and other sequences of interest will be apparent to those of ordinary skill in the art. Therefore, the foregoing discussion is intended to be exemplary rather than exhaustive.

Selectable Markers

Transformation usually produces a mixture of relatively few transformed cells and many more non-transformed cells. It is necessary to select for the transformed cells only. The transformed cells require a selectable marker that provides these cells with resistance to a selection agent such as an herbicide or antibiotic. The cells without this selectable marker die or their growth is significantly arrested. This method of selection is often referred to as negative selection.

Another method of selection is the use of certain auxotrophic markers that can compensate for an inability to metabolize certain amino acids, nucleotides, or sugars. This method requires the use of suitably mutated strains that are deficient in the synthesis or utility of a particular biomolecule, and the transformed cells are cultured in a medium that allows only cells containing the plasmid to grow. This method of selection is often referred to as positive selection.

Possible selectable markers resulting in negative selection of transformants and for use in connection with the present invention include, but are not limited to, a bar gene which codes for bialaphos resistance (Thompson et al., 1987) and; a gene which encodes an altered EPSP synthase protein (Steinrücken and Amrhein, 1980), thus conferring glyphosate resistance; a mutated PPO gene which confers butafenacial resistance, a nitrilase gene such as bxn from Klebsiella ozaenae which confers resistance to bromoxynil (Stalker et al., 1988); a mutant acetolactate synthase gene (ALS) which confers resistance to imidazolinone, sulfonylurea or other ALS-inhibiting chemicals (European Patent Application 154,204, 1985); a methotrexate-resistant DHFR gene (Thillet et al., 1988); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0,218,571, 1987). By way of example, a transformation method using the bar gene as the selectable marker could use bialaphos or glufosinate as a negative selective agent in the selection media during the transformation selection process.

An illustrative embodiment of a selectable marker gene capable of being used in systems to select transformants are the genes that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Where one desires to employ a bialaphos resistance gene in the practice of the invention, a particularly useful gene for this purpose is the bar or pat genes obtainable from species of Streptomyces (e.g., ATCC No. 21,705). The cloning of the bar gene has been described (Murakami et al. 1986; Thompson et al. 1987) as has the use of the bar gene in the context of plants other than monocots (De Block et al. 1987; De Block et al. 1989).

Selectable markers include a tetracycline resistance or an ampillicin resistance gene.

Selection markers resulting in positive selection, such as a phosphomannose isomerase gene, as described in patent application WO 93/05163, may also be used. Alternative genes to be used for positive selection are described in WO 94/20627 and encode xyloisomerases and phosphomanno-isomerases such as mannose-6-phosphate isomerase and mannose-1-phosphate isomerase; phosphomanno mutase; mannose epimerases such as those which convert carbohydrates to mannose or mannose to carbohydrates such as glucose or galactose; phosphatases such as mannose or xylose phosphatase, mannose-6-phosphatase and mannose-1-phosphatase, and permeases which are involved in the transport of mannose, or a derivative, or a precursor thereof into the cell. Transformed cells are identified without damaging or killing the non-transformed cells in the population and without co-introduction of antibiotic or herbicide resistance genes. In the past, it was shown that the positive selection method is often more efficient than traditional negative selection.

The present invention is directed to selection medias and methods directed to increasing the transformation efficiency when using negative selection agents, such as herbicides or antibiotics.

Several technologies for the introduction of DNA into cells are well known to those of ordinary skill in the art and can be divided into categories including but not limited to: (1) chemical methods; (2) physical methods such as microinjection, electroporation and particle bombardment; (3) viral vectors; (4) receptor-mediated mechanisms; and (5) Agrobacterium-mediated plant transformation methods.

After the construction of the plant transformation vector or construct, the nucleic acid molecule, prepared as a DNA composition in vitro, is introduced into a suitable host such as E. coli and mated into another suitable host such as Agrobacterium, or directly transformed into competent Agrobacteria. These techniques are well-known to those of ordinary skill in the art and have been described for a number of plant systems including but not limited to corn (maize), soybean, rice, sugar beet, cotton, and wheat.

Those of ordinary skill in the art will recognize the utility of Agrobacterium-mediated transformation methods. Representative strains can include, but are not limited to, Agrobacterium tumefaciens strain C58, a nopaline strain that is used to mediate the transfer of DNA into a plant cell; octopine strains, such as LBA4404; or agropine strains, e.g., EHA101, EHA105, or EHA109. The use of these strains for plant transformation has been reported, and the methods are familiar to those of ordinary skill in the art.

The present invention can be used with any one or more regenerable cell or tissue. Those of ordinary skill in the art recognize that regenerable plant tissue generally refers to tissue that after insertion of exogenous DNA and appropriate culture conditions can form into a differentiated plant. Such tissue can include, but is not limited to, callus tissue, hypocotyl tissue, cotyledons, meristematic tissue, roots, and/or leaves. For example, regenerable tissues can include calli or embryoids from anthers, microspores, inflorescences, and/or leaf tissues. Other tissues are also envisioned to have utility in the practice of the presently disclosed subject matter, and the desirability of a particular explant for a particular plant species is either known in the art or can be determined by routine screening and testing experiments after a review of the presently disclosed subject matter, whereby various explants are used in the transformation process and those that are more successful in producing transgenic plants are identified.

Once the regenerable plant tissue is isolated, the genetic components can be introduced into the plant tissue. This process is also referred to herein as “transformation”. The plant cells are transformed and each independently transformed plant cell is selected. The independent transformants are referred to as plant cell lines or “events”.

Methods for transforming dicots, primarily by use of Agrobacterium tumefaciens, and obtaining transgenic plants have been published for a number of crops including cotton, soybean, Brassica, and peanut.

Successful transformations of monocotyledonous plants describing the use of electroporation, particle bombardment, and/or Agrobacterium based methods have also been reported. Transformation and plant regeneration have been achieved and reported at least in asparagus, barley, maize, oat, rice, tall fescue, wheat, and sugarcane.

The present invention finds use in Agrobacterium-mediated transformation processes. Agrobacterium-inoculated explants are typically cultured on an appropriate co-culture medium to allow for transfer of the genetic component containing the gene-of-interest to be introduced into the plant cells/tissue for incorporation into its genome. Appropriate co-culture media is typically known for each culture system or can be determined by one of ordinary skill in the art. In accordance with the present invention, the co-culture media contains an effective amount of a compound or compounds which provide a carbohydrate energy source, such as sucrose, glucose, fructose, galactose, maltose, and dextrose.

Agrobacterium-inoculated explants are typically cultured on an appropriate medium containing an agent to inhibit Agrobacterium growth. This media is usually referred to as a delay media. The Agrobacterium-inoculated explants are cultured on such a media generally from one to fourteen days, preferably from two to seven days. Those of ordinary skill in the art are aware of the appropriate media components to inhibit Agrobacterium growth. Such media components include, but are not limited to, antibiotics such as carbenicillin or cefotaxime.

After the culture step to inhibit Agrobacterium growth, and optimally before the explants can be placed on selective media, they can be analyzed for efficiency of DNA delivery by a transient assay that detects the presence of a gene contained on the transformation vector, including, but not limited to, a marker gene such as the gene that codes for β-glucuronidase (GUS). The total number of blue spots (indicating GUS expression) for a selected number of explants is used as a positive correlation of DNA transfer efficiency.

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npt II) can be associated with the expression cassette to assist in breeding.

The present invention may be used for transformation of any plant species, including, but not limited to, corn (Zea mays), Brassica sp. (e.g., B. napus, B. rapa, B. juncea), particularly those Brassica species useful as sources of seed oil, such as canola, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. For purposes of this invention, plants include unicellular and multicellular algae, and include prokaryotic cyanobacteria.

Vegetables that may be used in accordance with the invention include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Leguminous plants include beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop.

Preferably, plants that may be transformed according to the present invention are crop plants, for example, corn, alfalfa, sunflower, Brassica, soybean, cotton, safflower, peanut, sorghum, wheat, oat, rye, millet, tobacco, barley, rice, tomato, potato, squash, melons, legume crops, e.g., pea, bean and soybean, and the like.

The present invention can include, after incubation on non-selective media containing the antibiotics to inhibit Agrobacterium growth without selective agents (delay medium). The explants are cultured on selective growth media including, but not limited to, a callus-inducing media containing a selective agent. Typical negative selective agents have been described and include, but are not limited to, chemicals such as glyphosate, phosphonthricyn or butafenacil. The plant tissue cultures surviving the selection media are subsequently transferred to a regeneration media suitable for the production of transformed plantlets. Selection and regeneration can be carried out over several steps.

The transformants produced are subsequently analyzed to determine the presence or absence of a particular nucleic acid of interest contained on the transformation vector. Molecular analyses can include, but are not limited to, Southern blots (Southern, Mol. Biol., 98:503-517, 1975), PCR (polymerase chain reaction), or TAQMAN® analyses. These and other well known methods can be performed to confirm the stability of the transformed plants produced by the methods disclosed. These methods are well known to those of ordinary skill in the art and have been reported (see, for example, Sambrook et al., Molecular Cloning: A Laboratory Manual, Second Edition, Cold Spring Harbor Laboratory Press, Cold Spring Harbor, N.Y., 1989).

The previous discussion is merely a broad outline of standard transformation and regeneration protocols. The tissue culture media can either be purchased as a commercial preparation or custom prepared and modified by those of ordinary skill in the art. Examples of such media would include, but are not limited to, Murashige and Skoog (Murashige and Skoog, Physiol. Plant, 15:473-497, 1962), N6 (Chu et al., Scientia Sinica 18:659, 1975), Linsmaier and Skoog (Linsmaier and Skoog, Physio. Plant., 18: 100, 1965), Uchimiya and Murashige (Uchimiya and Murashige, Plant Physiol. 15:473, 1962), Gamborg's media (Gamborg et al., Exp. Cell Res., 50:151, 1968), D medium (Duncan et al., Planta, 165:322-332, 1985), McCown's Woody plant media (McCown and Lloyd, HortScience 16:453, 1981), Nitsch and Nitsch (Nitsch and Nitsch, Science 163:85-87, 1969), and Schenk and Hildebrandt (Schenk and Hildebrandt, Can. J. Bot. 50:199-204, 1972).

### EXAMPLES

The following examples further illustrate the presently disclosed subject matter. They are in no way to be construed as a limitation in scope and meaning of the claims.

### Example 1

### Maize Transformation Using Differing Amounts of Sucrose in Transformation Selection Media

Transformation of immature maize embryos is performed essentially as described in Negrotto et al., (2000) Plant Cell Reports 19: 798-803. Various media constituents described therein can be substituted. For example, the selectable marker gene and the selection agent can be modified, as in the examples below.

### Transformation Plasmids and Selectable Marker

The genes used for transformation are cloned into a vector suitable for maize transformation as described above. Vectors used contain the either the PAT gene or the EPSPS gene as a selectable marker. PAT confers resistance to bialaphose, and EPSPS confers resistance to glyphosate.

Preparation of Agrobacterium tumefaciens: Agrobacterium strain LBA4404 (pSB1) containing the plant transformation plasmid is grown on YPC (yeast extract (5 g/L), peptone (10 g/L), NaCl (5 g/L), CaCl2.2H2O (1 g/L), 15 g/l agar, pH 6.8) solid medium for 2 to 4 days at 28° C. Approximately 0.8×10⁹ Agrobacteria are suspended in inoculation medium supplemented with 100 μM acetosyringone (As) Bacteria are pre-induced in this medium for about 20 to 240 minutes.

Inoculation: Immature embryos from are excised from 8-12 day old ears after pollination. The isolated mixture of endosperms with immature embryos are suspended in the inoculation medium and poured through a 1500 um sieve after shaking. The immature embryos are captured on a 380 um sieve after pouring the suspension through. Immature embryos are gently removed from the sieve and suspended in Agrobacterium suspension on co-cultivation medium using a sterile scalpel blade after 5 min 45° C. heat shock. The immature embryo suspension is spread evenly on the co-cultivation medium and the excess Agrobacterium suspension is removed using a sterile transfer pipette and filter papers. The immature embryos are then spaced and flipped with round scutellum side up and then cultured in the dark for two to three days. Subsequently, between 20 and 45 embryos per petri plate are transferred to the medium AW5Dicamba100Ti and cultured in the dark for 28° C. for 10-14 days for callus induction.

### Bialaphos/Pat Selection of Transformed Maize Cells

Following co-cultivation and callus induction, the transformed calli are step-wise selected on MS medium or MS medium containing alternating concentrations of sucrose (5, 10 and 20, g/l) and bialaphos (5 and 7.5 mg/l) as indicated in Table 1. The controls comprise calli selected on MS medium supplemented with 20 g/l sucrose and 5 or 7.5 mg/l bialaphos for 4 to 6 weeks at 2 to 3 week intervals.

In a first selection period referred to in Table 1 as 51, about 10-20 calli are transferred from callus induction medium comprising 30 g/L sucrose onto 51 medium MS medium contained 5 g/l sucrose and 7.5 mg/l bialaphos and cultured for approximately 2 weeks at 28° C. in the dark. Each callus was originated from an immature embryo. For the second selection period, referred to in Table 1 as S2, about 9 to 10 calli of 51 are transferred to each plate containing S2 medium comprising 10 g/l sucrose and 7.5 mg/l bialaphose and then cultured for approximately 2 weeks at 28° C. in dark. For the third selection period referred to as S3, 9 to 10 calli are transferred from each S2 plate to S3 medium contained 20 g/l sucrose and 5 mg/l bialaphos and cultured for approximately 2 weeks at 28° C. in dark.

In another embodiment of the invention, in the first selection period S1 about 10 to 20 calli from each plate were transferred from callus induction medium comprising 30 g/L sucrose onto S1 medium containing 10 g/L of sucrose and 7.5 mg/l bialaphos and cultured for approximately 3 weeks at 28° C. in the dark. Each callus was originated from an immature embryo. 9 to 10 calli per plate were transferred from the S1 medium to S3 medium containing 20 g/L of sucrose and cultured for 2 weeks at 28° C. in dark.

Regeneration of Transformed Plants: Bialaphos-resistant embryogenic callus were selected under dissecting microscope and 4 embryogenic calli per plate were transferred to regeneration medium supplemented with 5 mg/l bialaphos and 20 g/L sucrose and culture for approximately 2 weeks at 28° C. in the dark. Each callus is originated from an immature embryo.

The cultures are then transferred to the light room for 14 days at 25° C. with a 16 hour photoperiod. Then transfer the regenerated cultures to regeneration medium and cultured under light for additional 7-14 days at 25° C. with a 16 hour photoperiod if necessary.

Rooting: When the regenerated shoots reach about 2 cm in size, they were transferred to a Greiner containing the rooting medium comprising 20 mg/L sucrose. Only one shoot per callus line were selected, and transplant in each Grainer. Plants were subsequently sampled for analysis.

| | | |
|---|---|---|
| Inoculation medium (pH 5.2) | | |
| LS (Linsmaier and Skoog 1965) Modified Major 10X | 100 | ml/liter (l) |
| LS micro 1000X | 1 | ml/l |
| MS (Murashige and Skoog) iron 200X | 5 | ml/l |
| Dicamba | 5 | mg/l |
| Sucrose | 68.5 | g/l |
| Glucose | 36 | g/l |
| Vitamin Mix | | |
| Callus induction medium | | |
| MS Basal Salt Mixture | 4.30 | g/liter (l) |
| Proline (C5H9NO2) | 1.38 | g/l |
| Sucrose (C12H22O11) | 30.00 | g/l |
| Diacamba | 5 | mg/ml |
| Gelzan | 2.40 | g/l |
| Ticarcillin | 100 | mg/l |
| Vitamin mix | | |
| Selection medium (S1) | | |
| MS Basal Salt Mixture | 4.3 | g/l |
| Proline (C5H9NO2) | 1.38 | g/l |
| Sucrose (C12H22O11) | 5.0 | g/l |
| Diacamba | 5 | mg/l |
| Gelrite | 2.4 | g/l |
| Ticarcillin | 200 | mg/l |
| Bialaphos | 7.5 | mg/l |
| Vitamin mix | | |
| Selection medium (S2) | | |
| MS Basal Salt Mixture | 4.3 | g/l |
| Proline (C5H9NO2) | 1.38 | g/l |
| Sucrose (C12H22O11) | 10.0 | g/l |
| Diacamba | 5 | mg/l |
| Gelrite | 2.4 | g/l |
| Ticarcillin (100 mg/ml) | 2 | ml/l |
| Bialaphos | 7.5 | mg/l |
| Vitamin mix | | |
| Selection medium (S3) | | |
| MS Salt Mix | 4.29 | g/l |
| Sucrose | 20.0 | g/l |
| Dicamba | 5.00 | mg/l |
| Gelzan | 2.40 | g/l |
| Proline | 288.0 | g/l |
| Bialaphos | 5.0 | mg/l |
| Ticarcillin | 200 | mg/l |
| Vitamin mix | | |
| Regeneration medium | | |
| MS Basal Salt Mixture | 4.3 | g/l |
| MS Vitamins 100X | 10 | ml/l |
| Sucrose | 20 | g/l |
| Kinetin | 1 | mg/l |
| Gelzan | 3 | g/l |
| Ticarcillin | 200 | mg/l |
| IAA | 0.25 | mg/l |
| Rooting medium | | |
| Basal Salt Mixture | 3.2 | g/l |
| Sucrose | 30 | g/l |
| Gelzan | 2.4 | g/l |
| Ticarcillin 100 mg/ml | 200 | mg/l |
| IAA | 0.25 | mg/l |
| NAA | 0.5 | mg/l |
| Vitamin mix | | |

DNA Analysis: The presence of the GOI was determined by ±PCR assay or by a Taqman copy number assay. The presence of the PMI selective marker was determined by a Taqman copy number assay. The presence of the spectinomycin resistance gene selective marker was determined by ±PCR assay.

TABLE 1
Effect of alternating sucrose levels in bialaphos selection media on transformation of maize immature embryos.

| | Sucrose (g/l) and culture duration in | | | | | | | | | Survival | Regeneration | |
|---|---|---|---|---|---|---|---|---|---|---|---|---|
| | selection medium^{a} | | | | | | | | Transformation | callus to | frequency | Excape |
| Treatment | S1 | days | S2 | Days | S3 | Days | construct | # explant | frequency (%) | regeneration | (%) | (%) |
| Control 1 | 20 | 14 | | | 20 | 14 | 17589 | 530 | 8.8 | 50.9 | 41.7 | 75.7 |
| Control 2 | 20 | 21 | | | 20 | 14 | | 320 | 7.0 | 71.9 | 42.8 | 77.0 |
| Control 3 | 20 | 14 | 20 | 14 | 20 | 14 | | 400 | 5.0 | 87.0 | 25.7 | 79.0 |
| 1 | 20 | 14 | 10 | 14 | 20 | 14 | | 576 | 7.6 | 72.8 | 44.5 | 57.4 |
| 2 | 5 | 14 | 10 | 14 | 20 | 14 | | 340 | 10.3 | 53.8 | 37.3 | 43.9 |
| Control 4 | 20 | 21 | | | 20 | 14 | 17629 | 1800 | 9.0 | 56.4 | 22.5 | 61.0 |
| Control 5 | 20 | 14 | | | 20 | 14 | | 500 | 11.6 | 63.6 | 25.8 | 72.3 |
| 3 | 5 | 14 | | | 20 | 14 | | 1905 | 9.1 | 54.0 | 23.8 | 39.8 |
| 4 | 10 | 14 | | | 20 | 14 | | 500 | 11.0 | 57.6 | 24.7 | 36.2 |
| Control 6 | 20^{b} | 14 | 20^{b} | 14 | 20 | 14 | | 700 | 7.6 | 65.5 | 37.3 | 11.8 |
| 5 | 10^{b} | 21 | | | 20 | 14 | | 850 | 16.9 | 28.7 | 66.8 | 8.0 |
| 6 | 5^{b} | 14 | 10^{b} | 14 | 20 | 14 | | 850 | 23.5 | 45.2 | 65.4 | 2.4 |

^{a}S1 and S2 medium: MS salt mixture with 5 mg/L bialaphos except those specifically indicated; S3 medium: MS salt mixture with 5 mg/L bialaphos.
^{b}S1 and S2: 7.5 mg/L Bialaphos used

Treatments included application of, S1, S2, and S3 selection media, wherein selection incubation periods and sucrose levels were varied within each of S1, S2, and S3. By way of example, in all of controls, 20 g/l of sucrose was used in all of selections S1, S2, and S3. Control 1 and 2 were first cultured in MS medium with 5 mg/l bialaphos for 14 days and 21 days respectively and then subcultured in MS medium with 5 mg/l bialaphos for another 14 days in S3. Control 3 was cultured in MS medium with 5 mg/l bialaphos for 28 days with one subculture (S1 and S2) and then subcultured in MS medium with 5 mg/l bialaphos for another 14 days (S3), whereas control 6 was first cultured in MS medium with 7.5 mg/l bialaphos for 28 days with one subculture (S1 and S2), and then subcultured in MS medium with 5 mg/l bialaphos for 14 days (S3). Control 4 and 5 were first cultured in MS medium with 5 mg/l bialaphos for 21 and 14 days (S1), respectively, and then subcultured in medium with 5 mg/l bialaphos for 14 days (S3). All treatments 1, 2, 3, 4, 5 and 6 used different amounts of sucrose in the transformation selection media in selection steps S1, S2 and S3 as shown.

The results show that when sucrose level was reduced from 20 g/L to 5 g/L in various selection media supplemented with 5 mg/L bialaphos (Treatment 1-4), the transformation frequency did not dramatically improved, but the frequency of escapes rate significantly reduced, which saved the labor to transfer the non-transgenic cultures

When sucrose level was reduced from 20 g/L to 5 mg/L in selection medium supplemented with 7.5/L biaplaphos (Treatment 6), the transformation frequency of independent stable transgenic events was significantly increased to 23%, plant regeneration frequency was improved from 37% to 65%, and the frequency of escapes was dramatically reduced from 11.7% in the control to 2.4%.

### Glyphosate/EPSPS Selection of Transformed Maize Cells

Following co-cultivation and callus induction, the transformed calli are step-wisely selected on MS medium or MS medium contained differing concentrations of sucrose (5, 10 and 20, g/l) and glyphosate (2 mg/l) as indicated in Table 2.

In one embodiment of the present invention, in an incubation selection period referred to in Table 2 as S1, 10-20 calli from callus induction medium were transferred onto selection medium S1 and cultured for approximately 2 weeks at 28° C. in the dark. Each callus is originated from an immature embryo.

For the selection period referred to as S2, about 9 to 10 calli cultured in selection step S1 were transferred to each plate containing selection medium S2 and then cultured for approximately 2 weeks at 28° C. in dark.

For the selection period referred to as S3, about 9 to 10 calli cultured in selection period S2 were transferred to selection medium of selection period S3 containing 20 g/l sucrose and 2 mM glyphosate. and cultured for approximately 2 weeks at 28° C. in the dark.

In another embodiment and referring to Table 2, about 10 to 20 calli per plate from callus induction medium were transferred onto selection medium of selection period S1. and cultured for approximately 2-3 weeks at 28° C. in the dark. Each callus was originated from an immature embryo. About 9 to 10 calli per plates were transferred to selection medium of selection period S2. and cultured for approximately 2 weeks at 28° C. in the dark.

Regeneration of Transformed Plants: Glyphosate-resistant embryogenic callus were selected under dissecting microscope and 4 embryogenic calli per plate were transferred to regeneration medium. and cultured for approximately 2 weeks at 28° C. in the dark. The cultures were subsequently transferred to a light room for 14 days at 25° C. with a 16 hour photoperiod.

### Rooting:

One shoot per callus line, were selected and two shoots were transplanted to a Greiner containing rooting medium when the regenerated shoots reach above 2 cm in size in regeneration medium.

TABLE 2
Effect of differing sucrose level in glyphosate selection media on transformation of maize immature embryos

| | | | | | | | | | | Survival | |
|---|---|---|---|---|---|---|---|---|---|---|---|
| | Sucrose [g/L] and culture duration in selection medium^{a} | | | | | | | | | callus to | Regeneration |
| | S1 | | S2 | | S3 | | | | Transformation | regeneration | frequency |
| Treatment | (sucrose) | days | (sucrose) | Days | (sucrose) | Days | Construct | # explant | frequency (%) | (%) | (%) |
| Control | 20 | 14 | 20 | 14 | 20 | 14 | 17421 | 727 | 4.1 | 82.8 | 5.0 |
| 1 | 20 | 14 | 5 | 14 | 20 | 14 | | 556 | 12.9 | 15.3 | 87.1 |
| 2 | 20 | 14 | 10 | 14 | 20 | 14 | 17589 | 340 | 22.1 | 26.5 | 86.7 |
| Control | 20 | 14 | 20 | 14 | | | 15779 | 562 | 4.8 | 80.6 | 7.9 |
| Control | 30 | 14 | 30 | 14 | | | | 500 | 4.4 | 90.0 | 5.6 |
| 3 | 5 | 7 | 20 | 14 | | | | 335 | 8.7 | 55.8 | 15.5 |
| 4 | 10 | 7 | 20 | 14 | | | | 330 | 9.7 | 44.5 | 21.8 |
| 5 | 20 | 7 | 5 | 7 | 20 | 14 | | 566 | 12.5 | 13.1 | 55.0 |
| 6 | 5 | 14 | | | 20 | 14 | | 820 | 9.2 | 19.7 | 48.7 |
| 7 | 10 | 14 | | | 20 | 14 | | 972 | 17.9 | 26.6 | 73.4 |
| 8 | 5 | 14 | 10 | 14 | 20 | 14 | | 820 | 7.1 | 8.9 | 79.5 |
| 9 | 20 | 14 | 5 | 14 | 20 | 14 | | 919 | 29.5 | 34.5 | 87.4 |
| 10 | 20 | 14 | 5 | 14 | 20 | 14 | | 500 | 23.4 | 28.4 | 83.8 |
| 11 | 20 | 14 | 10 | 14 | 20 | 14 | | 1064 | 18.3 | 22.1 | 86.4 |
| 12 | 20 | 14 | 10 | 14 | 20 | 14 | | 397 | 13.1 | 15.9 | 85.7 |
| 13 | 30 | 14 | 5 | 14 | 20 | 14 | | 370 | 14.1 | 32.2 | 43.7 |
| 14 | 30 | 14 | 10 | 14 | 20 | 14 | | 359 | 15.3 | 29.5 | 52.8 |
| 15 | 5 | 14 | | | 20 | 14 | | 385 | 9.4 | 15.6 | 75.0 |
| 16 | 5^{b} | 14 | | | 20 | 14 | | 350 | 11.7 | 18.9 | 69.7 |
| 17 | 10^{b} | 14 | | | 20 | 14 | | 350 | 8.6 | 12.6 | 77.3 |

^{a}S1 and S2 medium: MS basal salt mixture with 2 mM glyphosate; 3 medium: MS salt mixture with 2 mM Glyphosate
^{b}Add 15 g/L sorbitol

By alternating level of sucrose in callus selections, transformation frequency of independent stable transgenic events was significantly increased from <5% (control) up to 29.5%. In addition, the amount of survival callus transferred from selection to regeneration was significantly reduced compared to control (>80%), which save a great deal of resources in labor and material. Plant regeneration frequency was also improved dramatically from <8% to >80%.

The method of the invention includes 3 consecutive 14 day incubation periods, wherein sucrose in the media is at 20 g/l in the first incubation period, at 5 g/liter in the second incubation period and 20 g/l in the third incubation period resulted in a transformation frequency of 29.5% compared to the control of under 5%, and a regeneration frequency of 87.4% compared to the controls having a regeneration frequency of under 8%.

The method of the invention also includes 2 consecutive 14 day incubation periods, wherein sucrose is at 10 g/l during first incubation period and at 20 g/l during the second incubation period.

As the data establishes, the invention encompasses a wide a range of reduced carbon content in the media. The invention includes protocols that vary the number of consecutive incubation periods and the incubation time for each.

| | | |
|---|---|---|
| S1 Selection Media | | |
| MS Basal Salt Mixture | 4.30 | g/l |
| Proline (C5H9NO2) | 1.38 | g/l |
| Sucrose (C12H22O11) | 5.00 | g/l |
| Diacamba | 5 | mg/l |
| Gelzan | 2.40 | g/l |
| Ticarcillin | 200 | mg/l |
| Glyphosate | 2 | mM |
| Vitamin mix | | |
| S2 Selection Media | | |
| MS Salt Mix | 4.29 | g/l |
| Sucrose | 20.00 | g/l |
| Dicamba | 5 | mg/l |
| Gelzan | 2.40 | g/l |
| Glyphosate | 2 | mM |
| Ticarcillin | 200 | mg/l |
| Vitamin mix | | |
| S2 Selection Media | | |
| MS Basal Salt Mixture | 4.30 | g/l |
| Proline (C5H9NO2) | 1.38 | g/l |
| Sucrose (C12H22O11) | 10.00 | g/l |
| Diacamba | 5.00 | mg/l |
| Gelzan | 2.40 | g/l |
| Ticarcillin | 200 | mg/l |
| Glyphosate | 2 | mM |
| Vitamin mix | | |
| RegenerationMedia | | |
| MS Basal Salt Mixture | 4.30 | g/l |
| Sucrose | 20.00 | g/l |
| Kinetin | 1 | mg/l |
| Gelzan | 3.00 | g/l |
| Ticarcillin 100 mg/ml | 200 | mg/l |
| IAA | 0.35 | mg/l |
| Vitamin mix | | |

### Example 2

### Sugarcane Transformation Using Differing Amounts of Sucrose in Transformation Selection Media

Various sugarcane (Saccharum) tissues can be used for generating transgenic plants. Additionally, a variety of sugarcane cultivars can be utilized (ARIEL D. ARENCIBIA et al., Transgenic Research 7, 213±222 (1998); Adrian Elliott et al., Aust. J. Plant Physiol. 25, 739-743; Z Wang, et al, J. Agricultural Biotechnology 2002, 10 (3) 237-240; S Zhang et al., J. Integrative Plant Biology 2006, 48(4):453-459; Shiromani et al Plant Cell Report 2011, 30: 439-448).

The method of the present invention includes embryogenic responses initiated and/or cultures established from sugar cane young leave rolls by culturing on callus induction medium. Established embryogenic cultures were weighted, and then inoculated and co-cultivated with the Agrobacterium tumefaciens strain EHA101 (Agrobacterium) containing the desired vector construction. Agrobacterium was cultured from glycerol stocks on solid YPC medium (100 mg/L spectinomycin and any other appropriate antibiotic) for about two days at 28° C. Agrobacterium is re-suspended in liquid MS-D2 medium. The Agrobacterium culture was diluted to an OD₆₀₀ of 0.3-0.4 and acetosyringone is added to a final concentration of 400 uM. Acetosyringone was added before mixing the solution with the sugar cane cultures to induce Agrobacterium for DNA transfer to the plant cells. For inoculation, the cultures were immersed in the bacterial suspension. The liquid bacterial suspension is removed and the inoculated cultures were placed on empty plate for co-cultivation and incubated at 22° C. for two days. The cultures were then transferred to callus induction medium with Ticarcillin (400 mg/liter) to inhibit the growth of Agrobacterium and cultured for 4-10 days at 28° C. in dark.

Referring to Table 3 below, for constructs utilizing the EPSPS selectable marker gene, cultures are transferred to selection S1 medium containing glyphosate as the selection agent and 400 mg/liter Ticarcillin and cultured for 3-4 weeks in the dark. For different treatments, the concentration of sucrose varied from 0 to 30 g/l and glyphosate level varied from 0.1 to 5 mM. In two step selection, highest transformation frequency is obtained by first culturing in S1 selection medium containing 5 g/l of sucrose and 2 mM glyphosate for 14 days, and then transferring to S2 selection medium containing 20 g/l sucrose and 1 mM glyphosate for 21 days. Resistant colonies were then transferred to regeneration medium, and grown in the dark for 7 days, and then moved to the light growth room for 14 days. Regenerated shoots were transferred Rooting media for 3-4 weeks and then moved to the greenhouse when they are large enough and have adequate roots. Plants are transplanted to soil in the greenhouse (To generation), and grown to maturity.

| | | |
|---|---|---|
| Callus Induction Media | | |
| MS basal salts | 4.3 | g/l |
| Sucrose | 30 | g/l |
| 2,4-D | 2 | mg/l |
| Phytablend | 7 | g/l |
| Vitamin Mix | | |
| S1 selection medium | | |
| MS basal salts | 4.3 | g/l |
| Sucrose | 5 | g/l |
| 2,4-D | 2 | mg/l |
| Glyphosate | 2 | mM |
| Ticarcillin | 400 | mg/l |
| Phytab lend | 7 | g/l |
| Vitamin Mix | | |
| S2 selection medium | | |
| MS basal salts | 4.3 | g/l |
| Sucrose | 20 | g/l |
| 2,4-D | 2 | mg/l |
| Glyphosate | 1 | mM |
| Ticarcillin | 400 | mg/l |
| Phytablend | 7 | g/l |
| Vitamin Mix | | |

TABLE 3
Effect of alternated sucrose levels in glyphosate selection
media on transformation of sugarcane callus

| | | | | | Explant | TF (events/g |
|---|---|---|---|---|---|---|
| | Sucrose (g/L) in selection medium | | | | fresh | fresh |
| Treatment | S1 | Days | S2 | Days | weight (g) | tissue %) |
| control^{a} | 30 | 21 | 30 | 21 | 4.2 | 0 |
| 1^{b} | 10 | 14 | 10 | 14 | 2.1 | 0.5 |
| 2^{b} | 15 | 14 | 15 | 14 | 1.4 | 0.7 |
| 3^{a} | 5 | 14 | 5 | 14 | 4.2 | 1.19 |
| 4^{b} | 5 | 14 | 5 | 14 | 2.1 | 2.4 |
| 5^{c} | 5 | 14 | 5 | 14 | 2.1 | 2.4 |
| 6^{d} | 5 | 14 | 20 | 21 | 2.8 | 5.71 |
| 7^{e} | 5 | 14 | 20 | 21 | 1.4 | 7.14 |

^{a}3 mM glyphosate
^{b}3 mM glyphosate
^{c}2 mM glyphosate
^{d}S1: 3 mM glyphosate; S2: 0.5 mM glyphosate
^{e}S1: 2 mM glyphosate; S2: 1 mM glyphosate

In the standard control wherein MS medium supplemented with 30 g/l sucrose and 3 mM glyphosate, no transgenic plants were recovered in several replications. When reduce sucrose concentration to 10 and 15 g/l, transformation frequency was improved and transgenic plants were able to be recovered. Transformation frequency was further improved to 2.4% per gram of fresh tissues when the sucrose concentration was reduced to 5 g/l in selection medium. Through application of altering sucrose level and adjusting proper concentration of glyphosate in step-wise selection, Transformation frequency was improved up to >7% per gram fresh tissue.

All references referred to in this document, including those documents in the References section are hereby incorporated by reference herein

### REFERENCES

Thompson, C. J., Movva, N. R., Tizard, R., Crameri, R., Davies, J. E., Lauwereys, M. and Botterman, J. Characterization of the herbicide-resistance gene bar from Streptomyces hygroscopicus. EMBO J. 6(9): 2519-2523, 1987
Steinrücken HC, Amrhein N. The herbicide glyphosate is a potent inhibitor of 5-enolpyruvyl-shikimic acid-3-phosphate synthase”. Biochem. Biophys. Res. Commun. 94 (4): 1207-12, 1980.
Perl, A., Lotan, O., Abu-Abied, M., and Holland, D. Establishment of an Agrobacterium-mediated transformation system for grape (Vitis vinifera L.): The role of antioxidants during grape-Agrobacterium interactions. Nature Biotechnol. 14(5):624-628, 1996;
Enriquez-Obregon, G. A., Vazquez-Padron, R. I., Prieto-Samsonov, D. L., Perez, M., and Selman-Housein, G. Genetic transformation of sugarcane by Agrobacterium tumefaciens using antioxidants compounds. Biotecnol. Application. 14:169-174, 1997;
Enriquez-Obregon, G. A., Vazquez-Padron, R. I., Prieto-Samsonov, D. L., de a Riva, G. A., and Selman-Housein, G. Herbicide-resistant sugarcane (Saccharum officinarum L.) plants by Agrobacterium-mediated transformation. Planta 206:20-27, 1998;
Enriquez-Obregon, G., A., Prieto-Samsónov, D., L., Riva, G., A. de la, Pérez, M., Selman-Housein, G., and Vázquez-Padron, R., I. Agrobacterium-mediated japonica rice transformation: a procedure assisted by an antinecrotic treatment. Plant Cell Tiss. Organ Cult. 59(3):159-168; 1999;
Frisch D. A., Harris-Haller L. W., Yokubaitis N. T., Thomas, T. L., Hardin S. H., Hall T. C. Complete Sequence of the binary vector Bin 19. Plant Molecular Biology 27:405-409, 1995;
Gustavo, A. R., Gonzalez-Cabrera, J., Vazquez-Padron, R., and Ayra-Pardo, C. Agrobacterium tumefaciens: a natural tool for plant transformation. Electronic J. Biotechnol. 1(3):118-133, 1998;
Das, D., Reddy, M., Upadhyaya, K., and Sopory, S. An efficient leaf-disc culture method for the regeneration via somatic embryogenesis and transformation of grape (Vitis vinifera L.). Plant Cell Rep. 20(11):999-1005; 2002;
Olhoft, P. M., Flagel, L. E., Donovan, C. M., and Somers, D. A. Efficient soybean transformation using hygromycin B selection in the cotyledonary-node method. Planta 216:723-735, 2003;
Dan, Y., Munyikawa, T. R. I., Kimberly, A. R., and Rommens, C. M. T. Use of lipoic acid in plant culture media. US Patent Pub. No.: US 2004/0133938 A1, 2004;
Dan, Y. A novel plant transformation technology, Lipoic acid. In Vitro Cell. Dev. Biol. Plant 42:18-A, 2006 (Abstract);
Dan, Y., Yan H., Munyikwa, T, Dong, J., Zhang, Y., and Armstrong, C. L. MicroTom, A High-Throughput Model Transformation System for Functional Genomics. Plant Cell Rep. 25:432-441; 2006;
Botterman, J and Leemans, J. Engineering Herbicide resistance in Plants. Trends in Genetics. 4(8):219-222, 1988;
Ishida et al. Nature Protocols, 2:1614-1621, 2007;
Dekeyser et al., Plant Physiol., 90:217-223, 1989;
Della-Cioppa et al., Bio/Technology, 5:579-584, 1987;
Da Silva, Evaluation of Carbon Sources as Positive Selection Agents, NZ Journal of Crop and Horticultural Science Vol. 32:55-67 (2004). Southern, Mol. Biol., 98:503-517, 1975;
Sambrook et al., Molecular Cloning: A Laboratory Manual, Second Edition, Cold Spring Harbor Laboratory Press, Cold Spring Harbor, N.Y., 1989;
Murashige and Skoog, Physiol. Plant, 15:473-497, 1962;
Chu et al., Scientia Sinica 18:659, 1975;
Linsmaier and Skoog, Physio. Plant, 18: 100, 1965;
Uchimiya and Murashige, Plant Physiol. 15:473, 1962;
Gamborg et al., Exp. Cell Res., 50:151, 1968;
Duncan et al., Planta, 165:322-332, 1985;
McCown and Lloyd, HortScience 16:453, 1981;
Nitsch and Nitsch, Science 163:85-87, 1969; and
Schenk and Hildebrandt, Can. J. Bot. 50:199-204, 1972.
Kumria, R., Waie, B., and Rajam M. V. Plant regeneration from transformed embryogenic callus of an elite indica rice via Agrobacterium. Plant Cell, Tissue and Organ Culture. 67(1): 63-71, 2001.
Sahoo, K. K., Tripathi, A. K., Pareek, A., Sopory, S. K., and Singla-Pareek, S. L. An improved protocol for efficient transformation and regeneration of diverse indica rice cultivars. Plant Methods. 7:49, 2011.
Jain, R. K., Davey, M. R., Cocking, E. C., and Wu, R. Carbohydrate and osmotic requirements for highfrequency plant regeneration from protoplast-derived colonies of indica and japonica rice varieties. J. Experimental Botany. 48(308):51-758, 1997.
Ren, J., Wang, G., and Yin, J. Dicamba and Sugar Effects on Callus Induction and Plant Regeneration from Mature Embryo Culture of Wheat. Agricultural Sciences in China. 9(1): 31-37, 2010.
Das, P., and Joshi, N. C. Minor modifications in obtainable Arabidopsis floral dip method enhances transformation efficiency and production of homozygous transgenic lines harboring a single copy of transgene. Advances in Bioscience and Biotechnology, 2:59-67, 2011.
Swedlund, B., and Locy, R. D. Sorbitol as the Primary Carbon Source for the Crowth of Embryogenic Callus of Maize Plant Physiol. 103: 1339-1346, 1993
Geng, P., La, H., and Wang, H., Stevens, E. J. C. Effect of sorbitol concentration on regeneration of embryogenic calli in upland rice varieties (Oryza sativa L.). Plant Cell, Tissue and Organ Culture. 92(3):303-313, 2008.
Xu, J., Wang, Y. Z., Yin, H. X., and Liu, X. J. Efficient Agrobacterium tumefaciens-mediated transformation of Malus zumi (Matsumura) Rehd using leaf explant regeneration system. Electronic J. Biotechnology. 12 (1):1-8, 2009.
Soo C. C. Influence of carbon sources on shoot organogenesis in Echinacea angustifolia DC. Life Science J. 10(3):1300-1303. 2013;
Joersbo, M., Jorgensen, K., and Brunstedt, J. A selection system for transgenic plants based on galactose as selective agent and a UDP-glucose:galactose-1-phosphate uridyltransferase gene as selective gene. Molecular Breeding. 11(4)315-323, 2003.
Godo, T., Matsui, K., Kida, T., and Mii, M. Effect of sugar type on the efficiency of plant regenerationfrom protoplasts isolated from shoot tip-derived meristematicnodular cell clumps of Lilium•hort. Plant Cell Reports 15:401-404, 1996.

What is claimed is:

1. A plant transformation selection media used to transform a plant cell, tissue or other suitable explant to generate a plant therefrom, comprising a carbohydrate energy source in the amount of 1 g/L to about 15 g/L and a negative selection agent in an amount effective to select for transformants, wherein said the carbohydrate energy source increases plant transformation frequency compared to the plant transformation frequency obtained when using a carbohydrate energy in the transformation selection media in an amount greater than 15 g/L.

2. The plant transformation media of claim 1, wherein the carbohydrate energy source is in the amount of 1.0 g/L to 10.0 g/L.

3. The plant transformation media of claim 1, wherein said carbohydrate energy source is sucrose.

4. The plant transformation media of claim 3, wherein the sucrose is in the amount of 1.0 g/L to 10 g/L.

5. The plant transformation media of claim 1, wherein the negative selection agent is a herbicide.

6. The plant transformation media of claim 1, wherein the negative selection agent is glufosinate in the amount of 0.5 mM to 3 mM.

7. The plant transformation media of claim 1, wherein the negative selection agent is bialaphos in the amount of 5 g/L to 7.5 g/L.

8. The plant transformation media of claim 1, wherein the negative selection agent is selected from the group bialaphos, glyphosate, butafenacial, bromoxynil, imidazolinone, sulfonylurea or other ALS-inhibiting chemicals, dalapon, and 5-methyl-trytophan, mesotrione and other HPPD-inhibitors.

9. The plant transformation media of claim 1, wherein the plant is a monocotyledonous plant.

10. The plant transformation media of claim 1, wherein the plant is a dicotyledonous plant.

11. The plant transformation media of claim 9, wherein the plant is a maize, rice, wheat, barley, sorghum, switch grass, turf grass, Poacea, or sugarcane plant.

12. The plant transformation media of claim 10, wherein the plant is a soybean, tomato, Brassica, cotton, cucurbitae, or sugarbeet plant.

13. A method of identifying a transformed plant cell comprising:
(a) isolating a explant suitable for transformation;
(b) combining the explant with a gene to produce transformed plant cells;
(c) culturing the transformed plant cells in a plant transformation selection media wherein the selection media contains a reduced level of carbohydrate energy source and a negative selection agent and incubating the cells for a period of time;
(d) transferring the cells incubated in step (c) to transformation selection media containing a negative selection agent and an amount of carbohydrate energy source greater than the carbohydrate energy source contained in the plant tissue culture media of step (c) and incubating the cells for a period of time;
(e) identifying the transformed cells incubated in the transformation selection media in step (d).

14. The method of claim 13, further comprising the step of regenerating at least one transformed cell to produce a transformed plant.

15. The method of claim 13, wherein the carbohydrate energy source is in the amount of 1 g/L to 10 g/L.

16. The method of claim 13, wherein the carbohydrate energy source is sucrose.

17. The method of claim 16, wherein the sucrose is in the amount of 1.0 g/L to 10 g/L.

18. The method of claim 13, wherein the plant transformation selection media includes 5 to 7.5 mg/L bialaphos.

19. The method of claim 13, wherein the plant transformation selection media includes 0.5 to 8 mM glyphosate.

20. The plant transformation media of claim 13, wherein the negative selection agent is selected from the group bialaphos, glyphosate, butafenacial, bromoxynil, imidazolinone, sulfonylurea or other ALS-inhibiting chemical, dalapon, 5-methyl-trytophan, mesotrione and other HPPD-inhibitors.

21. The method of claim 13, wherein said plant is a monocot.

22. The method of claim 21, wherein the plant is a maize, rice, wheat, barley, sorghum, switch grass, turf grass, Pocea, or sugarcane plant.

23. The method of claim 13, wherein said plant is a dicot.

24. The method of claim 23, wherein the plant is a soybean, tomato, Brassica, cotton, cucurbitae, or sugarbeet plant.

25. A method of producing a transformed plant comprising:
(a) isolating a explant suitable for transformation;
(b) combining the explant with a gene to produce transformed plant cells;
(c) culturing the transformed plant cells in a plant transformation selection media wherein the selection media contains a reduced level of carbohydrate energy source and a negative selection agent and incubating the cells for a period of time;
(d) transferring the cells incubated in step (c) to transformation selection media containing a negative selection agent and an amount of carbohydrate energy source greater than the carbohydrate energy source contained in the plant tissue culture media of step (c) and incubating the cells for a period of time;
(e) identifying the transformed cells incubated in step (d); and
(f) regenerating at least one transformed cell identified in step e to produce a transformed plant.

26. The method of claim 25, wherein the negative selection agent is selected from the group bialaphos, glyphosate, butafenacial, bromoxynil, imidazolinone, sulfonylurea or other ALS-inhibiting chemical, dalapon, 5-methyl-trytophan, mesotrione and other HPPD-inhibitors.
